(19) United States
(12) Patent Application Publication
(10) Pub. No.: US 2017/0228513 A1
(43) Pub. Date: Aug. 10, 2017

(54) PATIENT QUEUE MANAGEMENT SYSTEM

(71) Applicant: YIHAN ZHANG, THORNHILL (CA)

(72) Inventor: YIHAN ZHANG, THORNHILL (CA)

(21) Appl. No.: 15/494,456

(22) Filed: Apr. 22, 2017

Related U.S. Application Data
(63) Continuation-in-part of application No. 14/090,023, filed on Nov. 26, 2013.

(51) Int. Cl. G06F 19/00; G06Q 30/02; A61B 5/0402; A61B 5/145; A61B 5/00; A61B 5/0205

(52) U.S. Cl. CPC G06F 19/3418; A61B 5/6801; A61B 5/02055; A61B 5/0402; A61B 5/14542; A61B 5/002; A61B 5/0022; A61B 5/742; G06Q 30/0281; G06F 19/322; A61B 5/021

(57) ABSTRACT

A service provider system 110 operates to receive and store initial patient vital sign information, and to use this vital sign information to assign an initial queue position to the patient. The patient wears a mobile vital sign monitoring device that detects vital patient sign information and periodically sends this information to the system 110, which uses the current and past vital sign information to determine whether the patient vital signs are within an acceptable clinical range, and if not, the system 110 operates to modify the position of the patient within the queue.

### FIELD OF THE INVENTION

The present invention relates to an automated, customer reception systems and to methods for determining a position of a customer in a queue.

### BACKGROUND

To effect an orderly, efficient and fair customer support process, some organizations that provide a service can require that a customer meet with a receptionist or customer registration clerk upon their arrival at a service location in order for the service provider to determine what type of service the customer requires, and to determine the level of urgency with which the service should be provided. Typically, customers seeking a service will position themselves at the end of an appropriate physical line, or in the event that the process is automated, the customer can take a numbered or coded ticket from a dispenser which assigns them to a particular position in a virtual line or queue in which they will wait to meet with registration clerk. The degree to which the customer is satisfied with their treatment during the time they wait to be registered, or during the time that they wait for services from the service provider, can have a significant impact as to whether or not the customer returns to the same service provider or seeks an alternative service provider. If the process leading up to the provision of a service is determined by the customer to be pleasant, then it is more likely that the customer will return again to that service provider for the service.

Customer management systems are available that operate to gather specific information from a customer (register a customer) prior to the customers arrival or at the time the customer arrives. This customer information gathered during the registration process can then be used by the system to position the customer in a correct virtual line (queue), and, depending upon the urgency with which the service is being requested, position the customer in an appropriate position within the queue. Then, after the customer arrives at the service provider location, they will, depending upon the number of customers which have already been assigned to their queue, wait for a greater or lesser period of time to be seen by a receptionist or by a service provider. Typically, during this waiting period, a customer is obligated to sit in or proximate to a waiting area so that they can determine, either audibly or visually, that they are next in line to be seen by the receptionist.

The present invention can be best understood by reading the specification with reference to the following figures, in which:

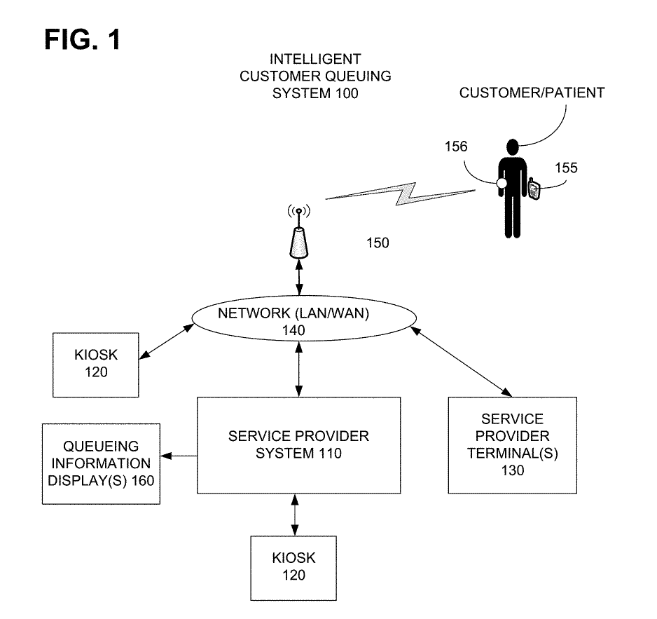

FIG. 1 is a diagram of an intelligent customer queuing system 100

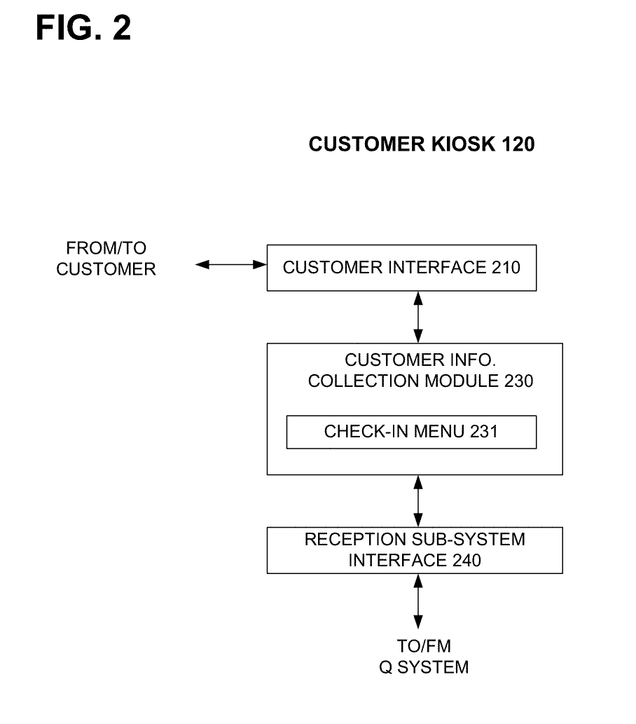

FIG. 2 is a block diagram illustrating the functional elements comprising a customer Kiosk 120.

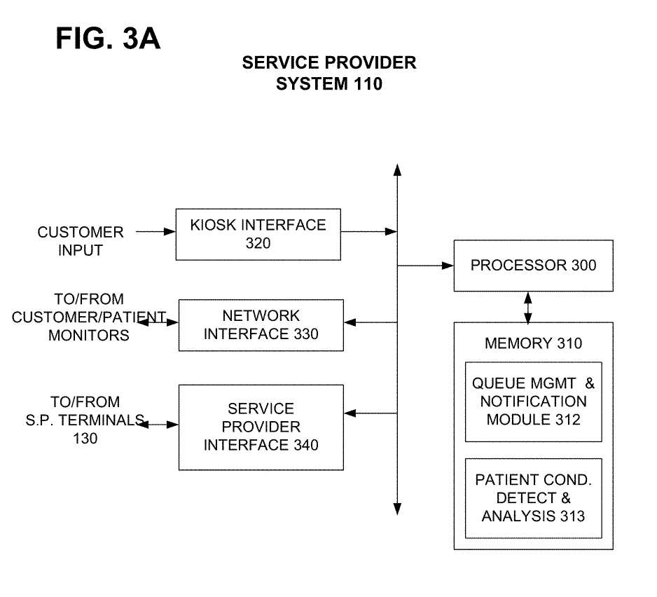

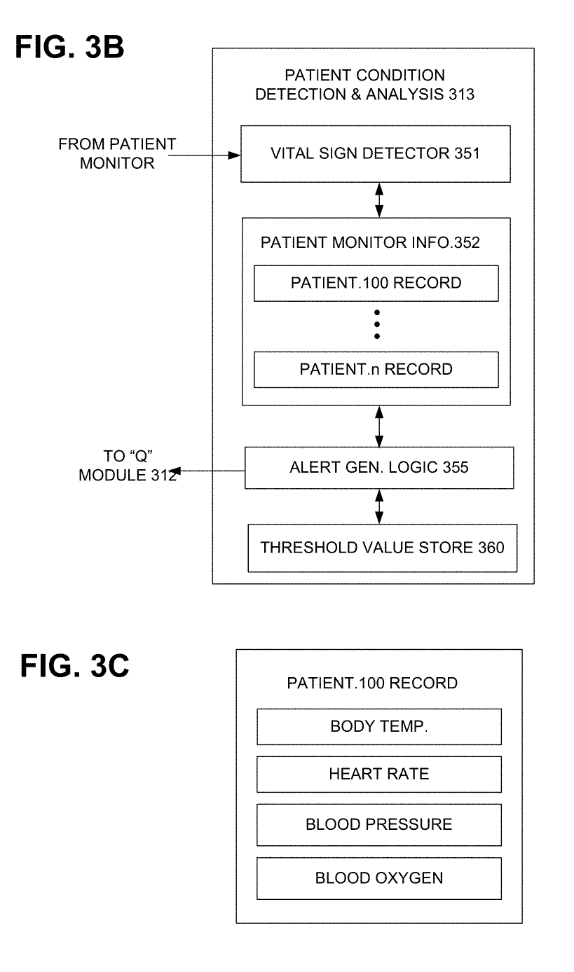

FIG. 3A is a block diagram illustrating the functional element comprising a service provider system 110.

FIG. 3B is a diagram showing functional elements comprising a patent condition detection & analysis module 313.

FIG. 3C shows the format of a patient record.

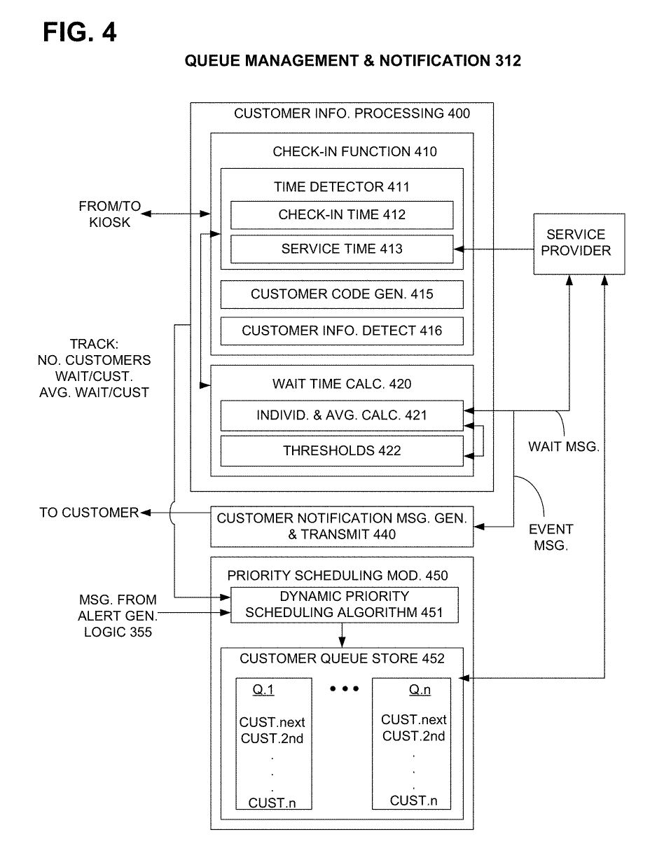

FIG. 4 is a block diagram illustrating function elements of a queue management and notification module 312.

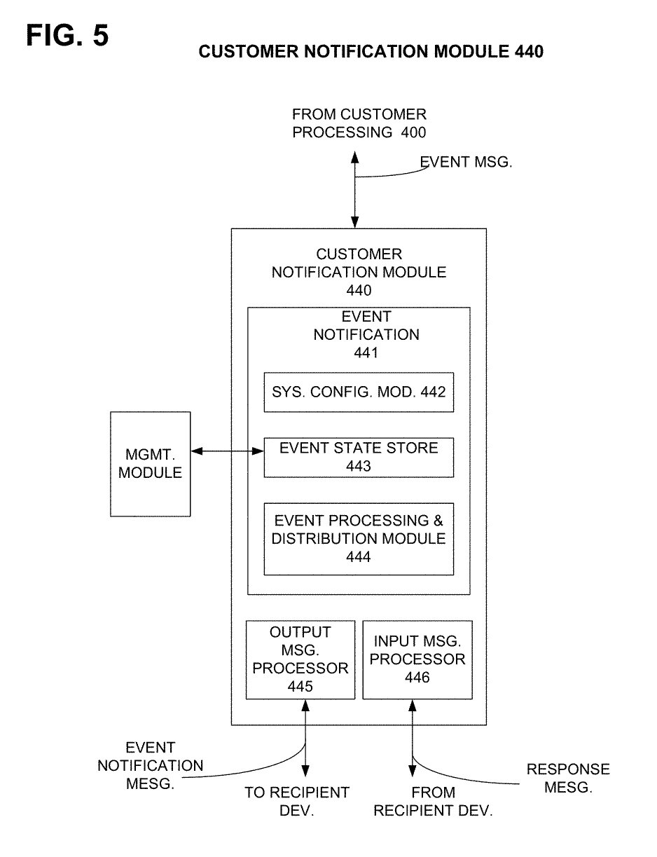

FIG. 5 is a block diagram illustrating functional elements comprising a customer notification module 440.

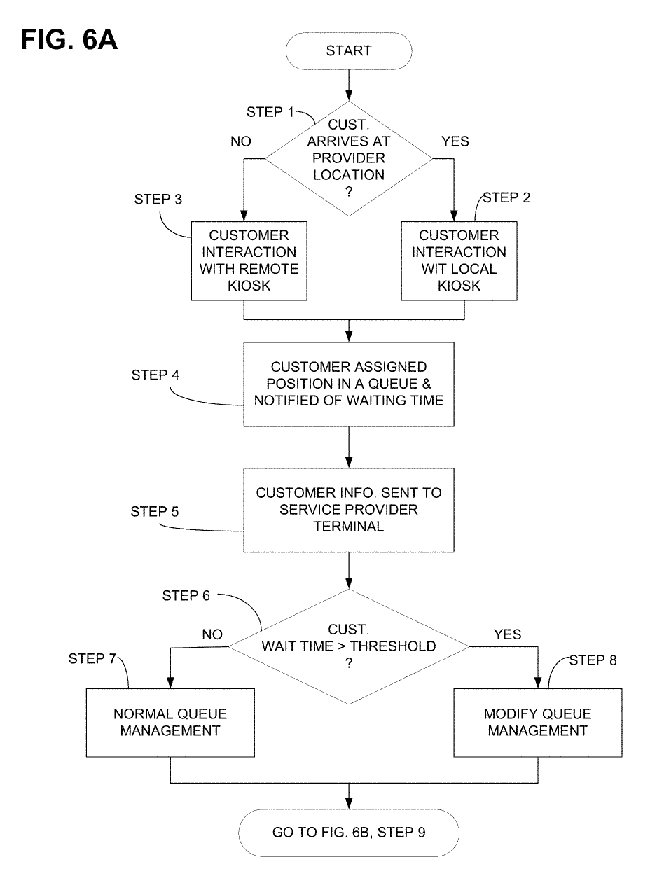

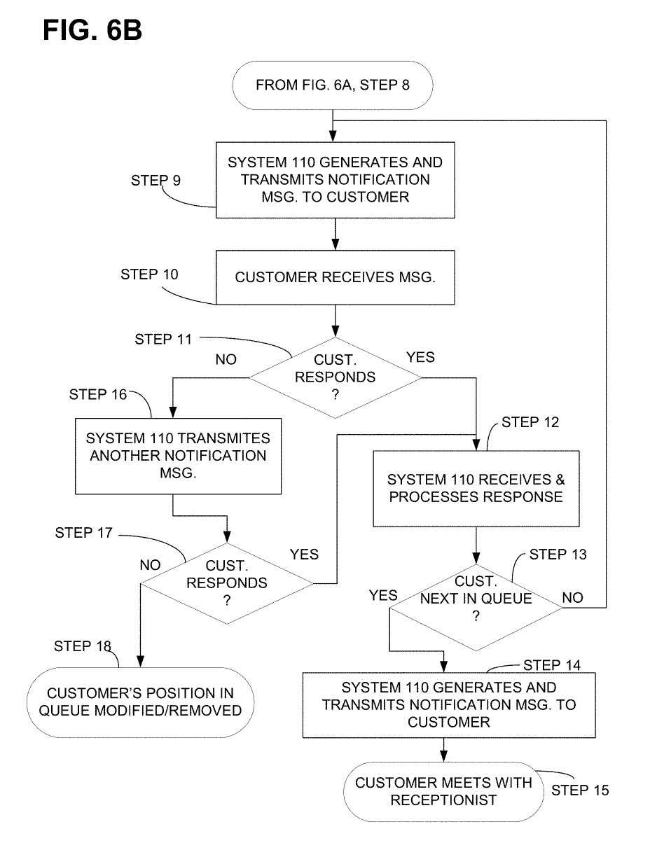

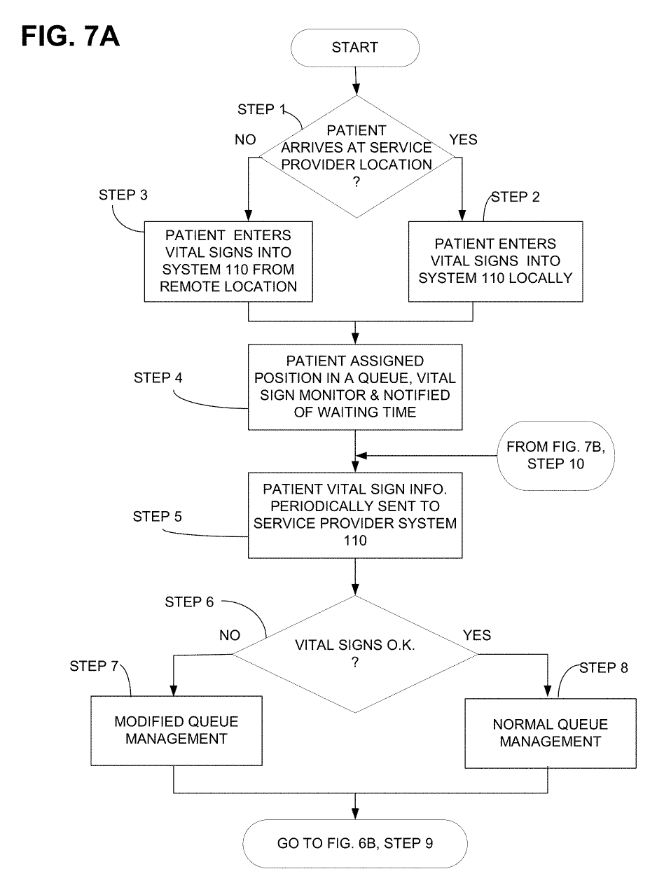

FIGS. 6A and 6B is a logical flow diagram illustrating the operation of the customer reception system.

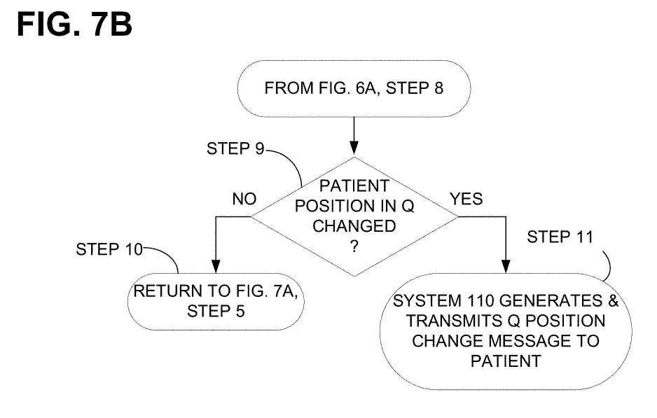

FIGS. 7A and 7B is a flow diagram illustrating the operation of the patient queue management module.

### CROSS REFERENCE TO RELATED APPLICATIONS

This application claims the benefit under 35 U.S.C. 120, 121, 365(c) or 386(c) of U.S. Non-Provisional patent application Ser. No. 14/090,023 entitled ““Intelligent Customer Queuing Network”, filed Nov. 26, 2013, which claims the benefit under 35 U.S.C. §119(e) of U.S. Provisional Patent Application Ser. No. 61/882,756 entitled “Intelligent Customer Queuing Network”, filed Sep. 26, 2013, and the entire contents of both applications are incorporated herein by reference.

### DETAILED DESCRIPTION

While current customer flow control systems attempt to engage waiting customers in some entertaining activity, such as watching television or by playing music, it is necessary that these customers not move away from either a display indicating their position in the queue or a public address system that announces the current status of the queue; otherwise, they may miss their opportunity to talk to a receptionist/registration agent, or worse, they may miss a notification that a service they are waiting for is ready for delivery. Therefore, it is desirable that a customer, who once assigned to a position in a queue, is able to move out of range of the functionality/means that notifies them of their current position in the queue, and it is desirable that a customer position in the queue is updated as customer information changes during the time they are in the queue.

In one embodiment, an intelligent customer queuing and notification system collects customer specific information that includes, among other things, information necessary to place the customer in a position within a queue and the network address of at least one customer communication device. The notification system can generate and transmit to a customer an alert message to one or more of the customer communication devices that has current queue position information associated with that customer. The queue position alert message can be transmitted to any communication device (mobile or stationary) designated by the customer as the device to which the queue position alert message is to be sent. The customer can designate a communication device to which the alert message is transmitted during the registration process or at any time prior to the registration process. The network address of the customer communication device to which the queue position alert message can be transmitted is stored at either or both of the customer flow control system and the customer notification system.

In another embodiment, the customer is a patient who can be instructed by the service provider to wear a physiological monitor or mobile vital sign monitoring system that is capable of wirelessly communicating current physiological or vital patient sign information to a service provider's network. The service provider can analyze this information to determine that the patient condition has changed since they entered the queue, and depending on the patent's current condition, advance or retard the patient's initial position in the queue to a current position in the queue.

FIG. 1 illustrates the component parts that can comprise an intelligent customer queuing system 100. Among other things, the system 100 operates to request information from a customer, receive information entered into the system by the customer, use this customer information assign the customer a position within a queue, and to send a message to the customer notifying them of their customer service times or other customer process information corresponding to them. Customer service times can include a time to physically be present in a queue to receive a service to be registered, it can be the period of time that they will have to wait after checking in to the start of a registration process, it can be the period of time from checking in to the end of the registration process, and/or it can be the entire time from checking in to the end of the registration process. According to the operation of the system 100, subsequent to initially checking in with the system and entering requested customer information, the customer is not required, and there is no need for the customer to remain physically proximate to the means by which the customer is notified of their current position in the queue. This notification means can be, but not is not limited to, audio notification via a loud speaker system or by an individual, or it can be a visual notification on a TV type screen. The system 100 is designed to send electronic messages to a customer via their preferable method (automated voice call, SMS, email or etc.) alerting them of pending service or registration times, thereby permitting the customer to roam away from their assigned queue or a customer waiting area. The customer position within a queue can be assigned and saved in a number of different formats. One format is an integer value which is a relative indication of a current customer queue position with respect to the top position in the queue, which can be “queue position 6” from the top position of 1, for instance. The position can be in the form of a running integer value that is assigned to each customer at the time they check-in. In this case, a first customer is assigned a number, 545 for instance, and the next customer is assigned a number, 546, and so forth.

The customer queuing system 100 of FIG. 1 is comprised of a number of operational elements. The central operation element of the system 100 is a service provider system 110 that is in communication over a local area network and/or over a wide area network 140 with one or more customer reception kiosks 120, one or more service provider terminals 130, with a plurality of customer communications devices 155 and physiological or mobile vital sign monitors 156 worn by a patient or customer. The service provider system 110 can be in communication with one or more displays 160 on which customers can view queuing information such as information about a ticket number being served or the average waiting time for each queue. The network 140 can be a Wi-Fi network, a DECT network or a wired Ethernet, or it can be the Internet or some other WAN or the network 140 can be any combination of these different network technologies. The particular network topology selected for implementation depends in large part on the type of organization implementing the customer system and/or the methods used to service the customers. The service provider system 110 is described later with reference to FIG. 3 and the customer kiosk is described later with reference to FIG. 2.

The customer communication devices 155 can be any type of wireless communication device such as a smart phone, a plain old telephone system (POTS) phone, tablet computer, a cellular phone or any other type of mobile communications device that is compatible with the local or public wireless communication protocols implemented by the system 100. The service provider terminals can be any type of computational device (server, PC, etc.) that permits a service provider to monitor, to configure and generally interact with the operation of the service provider system 110. From a terminal, the service provider can configure the system 110 to request that a customer enter certain personal information into the system during a check-in time, or the service provider can monitor the progress of a customer through the entire servicing process or monitor the average queue waiting time in case new service window needs to be opened to reduce the waiting time. From their terminal, the service provider can also manage the reception personnel depending upon customer queue time information they receive from the system 100.

FIG. 2 is a diagram illustrating several functional elements comprising a customer kiosk 120. It should be understood that this kiosk can be connected to the reception sub-system 110 over a LAN or over a WAN. The kiosk 120 has a customer interface element 210, a customer information collection module 230, and a reception sub-system interface 240. The customer information collection module 230 is comprised of an interactive customer check-in menu 231 that prompts a customer to select options displayed on a screen, prompts the customer to type or swipe their service card information into the system 100, prompts them to enter certain customer information, such as their name, weight, height, and prompts them to enter customer service requirements such as their service provider with which they have an appointment or would like an appointment, and any other information that may be pertinent to the service process.

FIG. 3A is a diagram illustrating the functional elements comprising the service provider system 110 described earlier with reference to FIG. 1. The system 110 can be any computational device, such as a network server, and is comprised of a processor 300 and associated memory 310, a kiosk interface 320, a network interface 330 and a service provider interface 340. The processor is any microprocessor device that is suitable to the demands of the system 110 and which executes logical instructions stored in the memory 310 to operate the system 110. The memory 310 also has a patient condition detection and analysis module 313 that, as described below in FIG. 3B, has functionality operating to detect and analyze information received from the vital sign monitors worn by patients.

FIG. 3B, shows the module 313 having a patient vital sign detector 351, a patient monitor information store 352 and alert generation logic 355. The detector 351 operates to receive telemetry information (vital sign information) from each patient monitor, to parse the information to determine what type of vital sign information comprises the telemetry, to determine which patient the telemetry is received from, and to store the patient information in the appropriate patient record comprising the patient monitor information 352. For example, a patent vital sign monitor associated with patient. 100 can periodically detect vital sign information and generate vital patient sign messages that include the identity of the patient or monitor device and patient physiological information (vital sign information) in the form of a current heart rate, blood pressure, blood oxygen level, EKG information, respiratory rate and body temperature information, to name only six. This information can then be stored, with a time stamp, in the patient. 100 record comprising the patient monitor information 352. History of the patient vital sign information can be stored in each record for use by the logic 355 to determine whether the patient's condition has changed since the time they checked in or from the time initial patient vital sign information is entered into the system. In this regard, the logic 355 can periodically examine each patient record to determine if there has been a change in the patient's clinical condition since the last time the record was examined, and if so, the logic can examine a store of threshold values to determine whether any vital patient sign information is currently outside an acceptable clinical range, such as whether the patient H.R. is above normal, or their blood pressure is below normal. In the event that the logic 355 determines that the vital signs of a patient fall outside a pre-determined range (i.e., blood pressure <50 or >180 mm HG), the logic 355 can then generate and send a queue priority update message to the queue management and notification module 312. The message can have instructions to move the patient to the top of the queue, or to advance the position of the patient in the queue some number of positions depending upon how far outside the range the vital signs are, or if more than one vital sign is outside an acceptable range.

FIG. 3C is a diagram showing four types of vital sign information associated with a patient record, patient. 100.

FIG. 4 is a diagram of the queue management and customer notification module 312 described earlier with reference to FIG. 3, and which is referred to hereinafter as the management module or the module 312. The management module 312 is illustrated to have a number of different functional modules to include, among other things, a customer information processing module 400, a customer notification module 440 and a dynamic, customer priority scheduling module 450. The dynamic, customer priority scheduling module 450 comprises a dynamic priority scheduling algorithm 451 and a store of customer queues 452 comprising a plurality of separate queues labeled Q.1-Q.n (where n is an integer value). The knowledge needed to design a dynamic, priority scheduling algorithm, such as the scheduling algorithm 451, is well known to those skilled in the art of designing scheduling systems and so will not be described here in any detail other than to mention that the design of the algorithm can be tailored according to customer need and the services being provided, or according to any other appropriate criteria. Generally, the scheduling algorithm is designed to operate on information supplied by a customer, a service provider and/or the patient condition detection and analysis module 313 to determine a prioritized position for each customer in a service queue.

Continuing to refer to FIG. 4, the customer information processing module 400 is illustrated to have two functional elements. It has a customer check-in function 410 and a customer waiting time calculation function 420. The customer check-in function 410 is, in turn, comprised of a time detector 411, a customer code generator 415 and a customer information detector 416. More specifically, the time detector 411 includes logical instructions that when executed by the processor 300 in FIG. 3 operate to detect a check-in time of a customer at a customer kiosk, for instance, and to detect the times at which a customer starts and completes a registration process. Check-in time logic 412 operates to detect a check-in time for a customer, and the service time logic 413 operates to detect the start and end times of a registration process. Alternatively, the service time logic 413 can operate to detect the times at which a service requested by a customer is started/provided and completed.

Subsequent to the time at which the customer checks-in at a service kiosk and enters their customer information, the customer code generator 415 calculates a unique code (2D bar code for instance) for the customer based on the customer information and the service they request, and the customer notification module 440 can dispense the code to the customer in the form or a paper ticket or electronically. The customer information detector 416 operates to receive information requested from the customer at the kiosk and discriminates between the different types of information entered by the customer. For instance, the customer can be prompted to enter their name, DOB, the customer's reason for requesting a service, the type of service they are requesting, the service provider's identity if known and other information that the system 110 can use to schedule the customer with a service provider. Depending upon the type of information a customer enters into the system 110, some or all of this customer information can be passed from the customer information processing module 400 to the priority scheduling module 450.

The check-in function 410 is in communication with the customer kiosk and generally operates to detect the time that each customer checks in and enters information requested by the system 110 and the times that they enter and exit a customer registration process. The total amount of time from check-in to exiting the registration process is referred to here as customer time. Subsequently to being detected, these times are passed to the wait time calculation function 420 which operates on the customer check-in times and the registration times (starting time of registration and/or ending time of registration) to calculate an estimated customer waiting time. According to one embodiment, the customer waiting time is the period of time from their checking in to the time that the registration process starts, and according to another embodiment, the waiting time it the time from check-in to the time the service they requested is provided, in another embodiment, the customer waiting time is the period of time from the customer checking in to the time the system 100 assigns the customer to the top position in their queue, the top position being the next customer to be served in the queue. In another embodiment, the customer waiting time is an actual estimated time at which the system will notify the customer that they are at the top of the queue (i.e., 1:30 PM). The calculated waiting time for each customer can be stored for later use in the memory 310 in association with the time calculation function 420. Further, the time calculation function 420 can calculate an average wait time for customers assigned to a particular queue (any one of queues Q.1 to Q.n) by summing all of the calculated wait times for each of the customers assigned to a queue and then dividing the resulting value by the number of customers assigned to the queue. Still further, the time calculation function 420 can also be assigned wait time threshold values 422 that are periodically examined by the time calculation logic 420 to determine if the wait times for individual customers or the average wait times exceed a threshold for each individual queue, and if the calculated wait time(s) exceed the specified time threshold stored in 422, then the time calculation function can operate to send a queue management message to the service provider. On the other hand, and regardless if the calculated customer wait time does or does not exceed the threshold value, a wait time message can be sent to the customer notification module 440 to update their current status (e.g. how many people are in front of them in the queue, etc.). A detailed description of the functional elements and the advantages in the operation of the module 440 is described later with reference to FIG. 5.

As described earlier, the dynamic, customer priority scheduling module 450 comprises a scheduling algorithm 451 and a store of some number of customer queues, Q.1-Q.n. Each customer queue, Q.1-Q.n can be dedicated to providing a different type or a different level of service. For instance, customers with less urgent service needs, or whose vital signs indicate that they are in relatively good clinical condition, can be assigned to the queue labeled Q.1, and customers with the most urgent need for service, or whose vital signs indicate that they are in relatively bad clinical condition, can be assigned to the queue labeled Q.n. The assignment of customers to queues by the dynamic priority scheduling algorithm 451 is determined by information entered into the system 100 by a customer, or is determined by information received from a vital patient sign monitor. Each queue, Q.1-Q.n, is illustrated to have n customer positions (with n being an integer value), labeled cust.next, cust.2nd-cust.n, and the scheduling algorithm can assign or reassign a customer to occupy any of the positions within a queue depending upon the urgency with which they are in need of a service associated with that queue, or depending upon a change in the customer/patients clinical condition.

According to one embodiment, the requested information is entered by a customer into a kiosk that is geographically proximate to the system 110. Geographically proximate in this context means that a customer kiosk is either in the same building or same group of buildings that are serviced by the system 110. According to another embodiment, a kiosk need not be geographically proximate to the system 110 to which it is in communication. In still another embodiment, the customer kiosk can be a triage kiosk capable to taking a customer's vital physical signs (blood pressure, temperature, blood oxygen) or other customer information employed by the system 100 to place the customer is a proper queue. Customer information received at a kiosk that is remote to the system 100 is transmitted over a communication network to the system 110. According to another embodiment, the patient information can be sent to the system 110 by a mobile monitor system worn by the patient.

FIG. 5 illustrates functional elements comprising the customer notification module 440. Generally, the customer notification module 440 operates to receive customer service information from the customer information processing module 400 comprising the system 100 and to determine whether or not to send the customer a message that includes information relating to a requested service and to determine what type of message to send. The customer notification module 440 can be tightly integrated into the system 110 or is can be a stand-alone module running on a server connected to the same network as system 110. The module 440 can be linked to only one system 110 or it can be linked to two or more systems substantially similar to the system 110. In addition to being in communication with the one or more service provider systems 110, the module 440 is configured to communicate over the network with a plurality of customer communication devices. The customer communication devices can be any of a variety of wired or wireless devices such as a mobile phone, a pager, a computational device, a wired phone system or any other device that is capable of establishing an electronic link with the module 440, and they can be connected to the notification module 440 over any suitable communication link. According to one embodiment of the invention, a customer device can, among other things, run an email client, and the transport mechanism between the module 440 and the customer device can be the Post Office Protocol (POP), the Simple Mail Transfer Protocol (SMTP), Internet Messaging Access Protocol (IMAP) or any other appropriate protocol.

The customer notification module 440 is comprised of a customer event notification module 441, a customer message processing module 445 and an input or response message processing module 446. The customer event notification module 441 can have a configuration module 442 that generally operates to receive configuration information from a management module via a graphical user interface and keyboard, for instance. The customer notification module 441 can also include means 443 for storing information associated with active customer messages, which means can include such things as whether or not a response to an outgoing customer message is received, the time that a customer message is generated and the time a response is received, for instance. The means 443 can be any computer readable medium suitable for storing the event message state information. The event notification module 441 can also include a customer information event processing and distribution module 444. Module 444 can operate on a customer information received from the system 100 to format an customer notification message. Module 444 can also operate on information received in a customer response message received at the input message processing module 446 to determine what further action the notification system 440 should take relative to the original customer notification message (such as send the response information to the system 100 which can determine to remove the customer from the queue or reposition the customer within the queue).

More specifically, module 444 operates to determine what customer service information, corresponding to information in an event message received from a system 110, is sent to the output customer message processor module 445 for inclusion in a customer notification message. It also operates to generate and assign a unique message identity to each event message receive from a system 110, and to send this unique message identifier to the output message processing module 445. The customer information comprising an event message generated by the module 444 can include, but is not limited to, an event identity, a customer identity, customer waiting time, customer time, customer processing time, message context, valid user responses, and time stamp. The unique message identifier includes information that refers to a particular version of a particular message. This unique message identifier can be generated by the event notification module 441 in any one or a number of ways which are well known to those skilled in the art, and so will not be described here. The module 444 also operates on information it receives from the input message processing module 446 to determine whether to escalate, cancel or to initiate some other action based upon information included in a response message.

With continued reference to FIG. 5, the output message processing module 445 generally operates to generate outgoing customer notification messages sent to one or more different customer devices, such as a pager, a wireless phone, a laptop or any other wired or wireless communications device that is configured to receive event notification messages from the notification system 440. Specifically, the processor 445 places information (From, Sender, reply-to, to, cc, subject) into one or more fields comprising a header of an event notification message according to any suitable standard or proprietary electronic messaging format, such as the messaging format described in the IETF RFC publication 5322 dated October, 2008 for instance. The processor 445 places a unique message identifier (UMI) into the body of the electronic message, and can place selected customer service information generated by the event notification module 441 into the body of the message.

The input message processing module 446 generally operates to parse input/response messages received from any of the plurality of customer devices in order to identify certain information included in the response message and to send this information to the customer processing module 400. Depending upon a customer's response to customer notification message, the customer processing module 400 can operate to update a customer's position in an assigned queue, delete the customer from the queue or transfer the customer to another queue. The logical operation of the system 110 to receive and process customer information and/or patient monitor vital sign information for the purpose of placing the customer in an appropriate initial of updated position in a particular queue, to notify the customer of their current or updated position with the particular queue, and to process customer responses to notification messages for the purpose of either repositioning the customer in the queue, transferring the customer to another queue or to deleting the customer from the queue will now be described with reference to FIGS. 6A and 6B.

In Step 1 in FIG. 6A, a customer either arrives at a service provider's physical location or can access via remote kiosk. If they are in a position to directly interact with a system kiosk, then in Step 2 the customer interacts with the system 110 through the interface 210 to enter information relative to their reason(s) for requesting the provider's service and any other information needed by the system 110 to place the customer into an appropriate queue. Alternatively, the customer can enter the same information as shown in Step 3 through a remote kiosk, not located proximate to the service provider's physical location. Regardless, the kiosk (remote or local) passes the customer information to the service provider system 110 and in Step 4 the system 110 uses this information to assign the customer a position in an appropriate queue. As described earlier with reference to FIG. 4, the system 110 maintains a plurality of queues into which it can place a customer. Typically, each queue can be configured to hold a different type of customer, or alternatively, every type of customer can be assigned to the same queue. After the customer is assigned a queue, they are notified by the system 110 of their position in the queue, the period of time they will probably have to wait before either seeing a receptionist of receiving a service, and any other information deemed by the system 110 necessary for the customer to know. The customer can be also notified directly by the kiosk with which they are interacting, or they can receive a notification message from the system 110 at any one of a number of different personal communication devices.

With continued reference to FIG. 6A, at Step 5 the information entered into the system 110 by the customer can be passed to a service provider terminal 130 for further processing, or to be viewed by a system administrator/service provider in order to monitor the customer service flow process. If in Step 6 the system 110 determines that an average waiting time for customers assigned to a particular queue exceeds a threshold value, then the system can send a message to the terminal 130 notifying the service provider of this condition. In Step 8, the service provider can manually change the customer's assignment to a different queue, can open another queue, or take any other action that has the effect of lowering the average waiting time for the customer. On the other hand, if the wait time in Step 6 is determined by the system 110 to be less than the threshold value, the system 110 operates to manage the assignment of customers to queues in the normal manner as in Step 7.

Subsequent to the customer being assigned a queue position, in Step 9 of FIG. 6B, the system 110 can generate and transmit a notification message to the customer informing them of their position in their assigned queue and the estimated period of time they will wait to either speak to a receptionist or receive a service. Alternatively, the customer can query the system 110 to receive their current queue position and/or the period of time they have to wait (or the actual time, which for example can be 3:30 PM May 3, 2013). In Step 10 the customer receives the notification message and may be prompted to respond. If in Step 11 the customer responds to the notification message, then in Step 12 the system 110 processes the response and proceeds to Step 13, where the system determines whether the customer position is next in the queue or not, and if so, in Step 14 the system 110 generates and transmits a message to the customer indicating that they are next up in the queue, and in Step 15 the customer proceeds to meet with a receptionist. If, on the other hand, the customer's position is not next in the queue, the process returns to Step 9 and the system can generate and transmit another message to the customer at a later time.

Referring back to Step 11, if the customer does not respond to the notification message sent in Step 9, then in Step 16 the system 110 can transmit another, second and alternatively subsequent notification messages or not, and if in Step 17 the customer does not respond to this message, then the system 110 in Step 18 can reposition the customer within the queue, remove the customer from the queue, or take some other appropriate action.

While customers find it convenient to roam around a location and have an alert message sent to them with an indication of their current or updated position in a queue, the customer notification system described in FIGS. 6A and 6B is not designed to alter the customer's queue positioned based on customer health information. According to another embodiment, a patient's queue position can be altered based on patient vital sign information detected by a mobile patient monitor and sent to the service provider system 110 for analysis. At the time a patient arrives at a service provider location (i.e., healthcare facility), and subsequent to entering initial vital patient sign information into the system 110, the patient can be assigned to wear a vital sign monitor. The system 110 can assign the patient an initial queue position based upon vital patient sign information entered upon arrival at the service provider location or vital sign received from the vital sign monitor by the service provider system 110. This initial position in a queue can be, maintained, advanced or retarded during the time that the patient is waiting for service depending upon changes in vital sign information sent to the system 110. This embodiment will be described below with reference to FIGS. 7A and 7b.

At Step 1 in FIG. 7A, a patient either arrives at a service provider's physical location or not. If they are local to the service provider system 110, at Step 2 they can directly interact with a system kiosk or provider receptionist to enter or provide their personal and vital sign information. Alternatively, at Step 3, the customer can enter the same information through a remote kiosk, not located proximate to the service provider's physical location. Regardless, the patient information is passed to the service provider system 110 and in Step 4 the system 110 uses this information to assign the customer an initial position in an appropriate queue. As described earlier with reference to FIG. 4, the system 110 maintains a plurality of queues into which it can place a customer. Typically, each queue can be configured to hold a different type of patient (i.e., cardiac patient, pulmonary patient, or other type of patient), or alternatively, every type of patient can be assigned to the same queue. After the patient is assigned a queue, they are notified by the system 110 of their position in the queue, the period of time they will probably have to wait before seeing a service provider (i.e., doctor or other healthcare professional), and any other information deemed by the system 110 necessary for the patient to know. The customer can be also be notified directly by the kiosk/receptionist with which they are interacting, or they can receive a notification message from the system 110 at any one of a number of different personal communication devices.

With continued reference to FIG. 7A, at Step 5 vital patient sign information is periodically detected by the vital sign monitor worn by the patient, and is transmitted in a vital sign message to the system 110. Specifically, the vital sign message is sent to the patient condition detection and analysis module 313 comprising the system 110, where the patient record is updated with the current vital sign information. The logic 355 comprising the analysis module 313 examines the patient record to determine if there has been a vital sign change since the last time the record was updated. If there has been a change, then at Step 6 the logic determines whether the change in vital sign is within an acceptable clinical range, and if not a patient condition update message is sent to the queue management and notification module 312, which at Step 7 may cause the patient position in a queue to be altered. If in Step 6 the system 110 determines that the patient vital sign(s) are within an acceptable clinical range, then the normal queue management scheme described with respect to FIGS. 6A and 6B can be employed.

Turning now to FIG. 7B, if at Step 9 it is determined that the patient queue position has changed, then the system 110 at Step 11 operates to generate and transmit a queue position change message to the patient, otherwise the process returns to FIG. 7A, Step 5.

It should be understood, that the mobile vital sign monitor worn by a patient can be configured to detect one or more vital patient signs. The monitor can be worn on the patient's wrist or worn as an item of clothing with the detectors strategically positioned to detect a vital sign of interest. The vital sign monitor can be designed to wirelessly communicate with the service provider system 110 using any appropriate wireless communication technology, whether it is long, medium or short range communication technology. Each mobile vital patient sign monitoring design is associated with a unique identifier, and this unique identifier is assigned (along with the monitor) to a patient.

The forgoing description, for purposes of explanation, used specific nomenclature to provide a thorough understanding of the invention. However, it will be apparent to one skilled in the art that specific details are not required in order to practice the invention. Thus, the forgoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications, they thereby enable others skilled in the art to best utilize the invention and various embodiments with various modifications as are suited to the particular use contemplated. It is intended that the following claims and their equivalents define the scope of the invention.

I claim:

1. A method for operating a queue management system, comprising:
receiving, by the queue management system, initial vital sign information associated with a patient;
assigning, the patient to an first position in a queue based upon the initial vital sign information;
periodically detecting, by a mobile vital patient sign monitor worn by the patient, current vital patient sign information and sending it to the queue management system;
determining, by the queue management system, that the clinical condition of the patient has changed based upon a comparison of the initial and the current vital patient sign information to be different; and
the queue management system operating to change the position of the patient in the queue from the first position to a second position based upon the changed clinical condition of the patient.

2. The method of claim 1, wherein the position of the patient in the queue is advanced if the clinical condition of the patient is determined to be outside an acceptable clinical range.

3. The method of claim 1, further comprising sending a message to the patient when their position in the queue changes.

4. The method of claim 1, wherein the mobile vital patient sign monitor is configured to detect one or more types of vital signs associated with the patient.

5. The method of claim 1, wherein the current vital sign information can be comprised of one or more of a heart rate, body temperature, respiratory rate, blood oxygen level, blood pressure, EKG information.

6. The method of claim 1, wherein the mobile vital patient sign monitor communicates wirelessly with the queue management system.

7. The method of claim 1, wherein the condition of the patient is determined to change if at least one of the same types of initial and current vital sign information is different.

8. A system for managing a patient position in a queue, comprising:
a mobile vital patient sign monitor worn by a patient which is in wireless communication with a queue management system, the monitor operating to periodically detect current vital sign information associated with the patient and to send the vital sign information to the queue management system which operates to compare the current vital sign information to initial vital patient sign information and change the position of the patient in the queue from an initial first position to a second position if it compares the initial and current vital sign information to be different.

9. The method of claim 8, wherein the position of the patient in the queue is advanced if the clinical condition of the patient is determined to be outside an acceptable clinical range.

10. The method of claim 8, further comprising sending a message to the patient when their position in the queue changes.

11. The method of claim 8, wherein the mobile vital patient sign monitor is configured to detect one or more types of vital signs associated with the patient.

12. The method of claim 8, wherein the current vital sign information can be comprised of one or more of a heart rate, body temperature, respiratory rate, blood oxygen level, blood pressure, EKG information.

13. The method of claim 8, wherein the mobile vital patient sign monitor communicates wirelessly with the queue management system.

14. The method of claim 8, wherein the condition of the patient is determined to change if at least one of the same types of initial and current vital sign information is different.
